(19) United States
(12) Patent Application Publication
(10) Pub. No.: US 2026/0174646 A1
(43) Pub. Date: Jun. 25, 2026

(54) Topical detergent composition comprising ascorbyl glucoside and ascorbyl palmitat

(71) Applicant: Mitochon S.r.l., San Lazzaro di Savena BO (IT)

(72) Inventors: Andrea FRATTER, San Lazzaro di Savena BO (IT); Silvia BUSCAROLI, San Lazzaro di Savena BO (IT); Ettore FEDERICI, San Lazzaro di Savena BO (IT)

(21) Appl. No.: 19/124,522

(22) PCT Filed: Oct. 31, 2023
(86) PCT No.: PCT/IB2023/060948

§ 371 Date: Apr. 25, 2025

(30) Foreign Application Priority Data
Nov. 7, 2022 (IT) 102022000022911

(51) Int. Cl. A61K 8/34; A61K 8/37; A61K 9/00; A61Q 19/08; A61Q 19/10

(52) U.S. Cl. CPC A61K 8/345; A61K 8/37; A61K 9/0014; A61Q 19/08; A61Q 19/10

(57) ABSTRACT

The present invention relates to atopical detergent composition comprising ascorbyl glucoside and ascorbyl palmitate, a washing and transcutaneous absorption-promoting base comprising at least one non-ionic surfactant, at least one anionic surfactant, glycerol, in combination with suitable excipients and/or diluents.

### FIELD OF THE INVENTION

The present invention belongs to the technical field of topical detergent compositions, and is based on ascorbyl glucoside and ascorbyl palmitate.

### BACKGROUND ART

Skin ageing is a composite process which includes various phenomena occurring in the dermo-epidermal region in response to endogenous and exogenous stimuli.

In particular, at the epidermal level, sun exposure, prolonged contact with pollutants and allergens, dehydration caused by aggressive cleansers on the inter-corneal lipid matrix or poor biosynthesis of barrier lipids (atopic dermatitis, psoriasis, etc.), as well as a state of chronic inflammation can contribute to accelerating skin ageing, also affecting the integrity of the dermal district. It is precisely in the dermal district, consisting of connective tissue rich in collagen and elastic fibres and endowed with an aqueous extracellular matrix, that the phenomena most closely linked to the onset of premature skin ageing develop.

The scientific community's attention is focused on the following phenomena with regard to skin ageing: reduced biosynthesis of connective and extracellular matrix linked to mitochondrial impairment (Defective Power House Model) [1]; fibroblast senescence with increased gene expression of matrix-degrading enzymes [2]; telomere shortening [3]; inadequately protected sun exposure [4, 5].

The various dermo-systemic approaches proposed in Aesthetic Medicine to reduce the development of skin ageing include the use of cosmetics containing dermo-trophic and antioxidant substances such as coenzyme Q10, ascorbic acid, ferulic acid, phloretin, various plant extracts rich in polyphenols, resveratrol, and others. Of these, the substance which has attracted the most interest over the years and has never suffered a decline in commercial success is ascorbic acid (AA). Ascorbic acid is an acid-functioning lactonic enediol (FIG. 1) which biologically plays a key role in the ageing of collagen fibres (procollagen) in the Rough Endoplasmic Reticulum (RER), upon activation of Lysine and Proline-hydroxylase, of which it is a cofactor, and through increased gene expression of neo-collagenesis enzymes [6].

Vitamin-derived ascorbic acid is extracted from several species of citrus fruit and is ubiquitously present, in various concentrations and synthetically derived, in many successful cosmetic products as well as in many food supplements. Thanks to this mechanism, ascorbic acid exerts a fundamental physiological function in maintaining the integrity of the epithelia, hence the occurrence of scurvy in deficient individuals (impaired integrity of mucous epithelia with gingival lesions and vascular endothelium). Precisely because of this fundamental function and its well-known antioxidant-reducing activity, this substance is widely used in cosmetics and nutraceuticals.

### PROBLEMS OF THE PRIOR ART

A first problem to address is that ascorbic acid, by virtue of the antioxidant properties thereof, is not very stable in cosmetic and integrative forms, especially when these involve an aqueous solution or even just the presence of water in the formulation.

In fact, ascorbic acid can be oxidised by the oxygen dissolved in water with the formation of dehydroascorbic acid, which is no longer functional until after re-hydrogenation (FIG. 2). In order to remedy the oxidation problems of the molecule, many ascorbic acid derivatives have been developed in recent years. In particular, the substances which have attracted the most interest are enediol hydroxyl phosphate derivatives: sodium and magnesium ascorbyl phosphate (FIG. 3). This chemical modification makes it impossible for the ascorbic radical to be oxidised by dehydrogenation, thus ensuring high stability in formulations.

On the other hand, such a structural modification does not solve the problem of transcutaneous penetration. In fact, a further limitation to the use of ascorbic acid is the percutaneous absorption thereof, which is hampered by the high hydrophilicity of the compound. Pinnell et al demonstrated that ascorbic acid can only penetrate the skin effectively if it is at a concentration at or slightly greater than 20% in the topical preparation and if its pH is below 3.5 (in general, below the pKA of ascorbic acid of 4.2) [7]. At the same time, it is also true that under these conditions, it is very difficult to formulate products which are safe, free of sensitising effects and at an acceptable cost. The ionised form of the molecule not only does not improve, but paradoxically worsens the transfer of ascorbic acid into the epidermis and dermis, since the transfer physiologically involves diffusion through regions with a high lipid component (skin barrier, membrane phospholipid bilayer) [8, 9].

Ascorbyl palmitate (AP), an ester of ascorbic acid with palmitic acid (FIG. 4), also initially aroused interest due to the high lipophilicity thereof, theoretically suitable for transcutaneous penetration, but studies have shown a reduced ability to release ascorbic acid into the epidermis [8,9], in addition to the fact that ascorbyl palmitate does not solve the problem of poor chemical stability [8,9]. Ascorbyl palmitate is used as an antioxidant and anti-peroxidant in the food, pharmaceutical and cosmetics industries (E 304). Other ascorbic acid derivatives, such as ascorbyl iso- and tetrapalmitate, possess properties substantially similar to those already discussed for ascorbyl palmitate.

In general, other factors which increase the rate of ascorbic acid degradation are: a high pH or temperature, the presence of dissolved oxygen and catalytic quantities of metal ions.

Therefore, although magnesium ascorbyl phosphate and ascorbyl palmitate are effective for oral use, they prove incapable of increasing tissue levels of ascorbic acid when applied to the skin [7].

Several strategies have been employed to reduce the phenomenon of ascorbic acid degradation in topical compositions, such as: reducing the amount of oxygen during preparation; oxygen-impermeable packaging; designing technological encapsulation of ascorbic acid; low pH compositions; minimising water; inclusion of electrolytes and other antioxidants. Further, skin treatment with techniques using lasers, or microdermabrasion or sonophoresis have been shown to greatly improve the topical release of vitamin C and derivatives which normally have poor dermal penetration [8].

Despite this, the stability of ascorbic acid remains a challenge, and some of these approaches may involve preparation difficulties and/or high costs.

Therefore, due to the above considerations, there is a need to design a topical composition capable, on the one hand, of solving the problem of ascorbic acid oxidation in topical forms, preferably liquid or when containing water; and on the other hand, of increasing transcutaneous ascorbyl permeation.

### SUMMARY OF THE INVENTION

The Applicant has developed a topical detergent composition comprising
(i) ascorbyl glucoside and ascorbyl palmitate,
(ii) a washing and transcutaneous absorption-promoting base comprising
   at least one non-ionic surfactant,
   at least one anionic surfactant,
(iii) glycerol,
in combination with suitable excipients and/or diluents.

### Advantages of the Invention

The topical detergent composition of the invention promotes an increase in the bioavailability of ascorbic acid thanks to a mechanism promoting the permeability of ascorbic acid precursors. The mechanism promoting the permeability of such precursors acts in parallel on several fronts:
the combination of the two active agents in the form of ascorbic acid precursors, i.e., ascorbyl glucoside (FIG. 5) and ascorbyl palmitate, promotes an increase in the bioavailability of ascorbic acid or ascorbate; specifically, the enediol hydroxyl of the ascorbyl glycoside is covalently bound with glucose, preventing oxidative dehydrogenation; this stabilises the molecule on the one hand, and promotes permeation through the stratum corneum (SC) on the other, since the molecule is not ionised. Thus the ascorbyl glucoside is hydrolysed within the epidermis through α-glucosidases [10] to generate ascorbic acid in situ [11, 12]. On the other hand, ascorbyl palmitate, by virtue of its amphiphilic and surfactant properties, known in the literature [13], can act both as an adjuvant to the detergent action discussed below, and as a secondary source of intraepidermal ascorbic acid.
the washing base is capable of increasing the permeation of ascorbic acid through the skin, by means of a mechanism of structural modification of inter-corneal lipids and the transient opening of aqueous channels within the epidermis, but without significantly altering the functionality of the stratum corneum (SC). Thereby, the Applicant converts a known ‘disadvantage’ related to the interaction of traditional surfactants with the components of the skin barrier, into a mechanism for promoting the transcutaneous passage of ascorbic acid precursors. Therefore, in addition to notoriously cleansing the skin surface, the detergent action also promotes the transcutaneous permeation of ascorbyl glucoside, ensuring a good intraepidermal diffusion and intrinsic chemical stability thereof.

It should further be noted that any excessive alteration of the structure and functionality of the skin barrier, induced by the washing base, is counterbalanced by means of:
a precise choice of surfactants and the amount thereof in the composition, thus obtaining a washing base in a form which is gentle or non-aggressive, but at the same time such as to favour the transcutaneous permeation of ascorbic acid precursors;
the essential presence of glycerol comprised in the composition of the invention which forms hydrogen bonds with water, reducing the thermodynamic activity thereof, and making the washing base less reactive towards skin barrier components.

The topical detergent composition is effective without having to resort to very high concentrations of ascorbic acid (e.g., close to or above 20%) and without the need to set the pH of the composition preferably to a value ≤3.5, below which the skin may be affected by irritative phenomena and alterations of the barrier and immune function [16].

Finally, the topical detergent composition is effective in promoting the absorption of ascorbic acid precursors and, thus, in increasing the bioavailability of ascorbic acid, even though it is a rinse-off composition (as opposed to leave-on compositions, such as serums or emulsions), i.e., intended for washing after application; such efficacy is of particular interest considering that, being a detergent, the composition does not require a stay (or setting) time to act.

### DESCRIPTION OF THE DRAWINGS

FIG. 1—Enediol-lactone structure of L-Ascorbic Acid;

FIG. 2—Oxidation and reduction reaction of L-Ascorbic Acid;

FIG. 3—Structure of Sodium Ascorbyl Phosphate;

FIG. 4—Structure of Ascorbyl Palmitate;

FIG. 5—Structure of Ascorbyl 2-Glucoside.

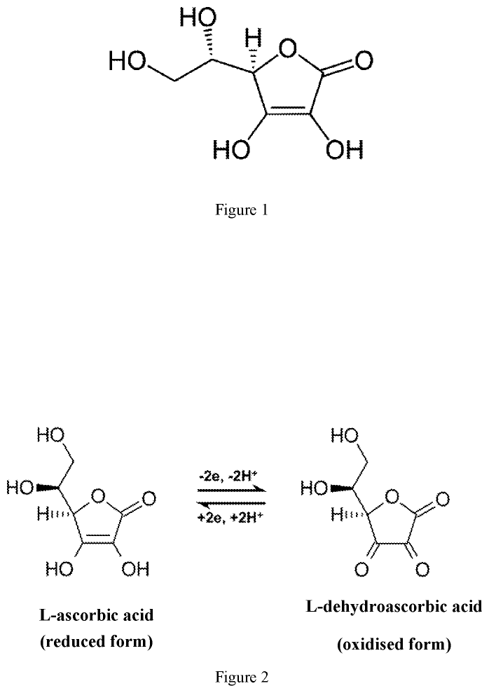

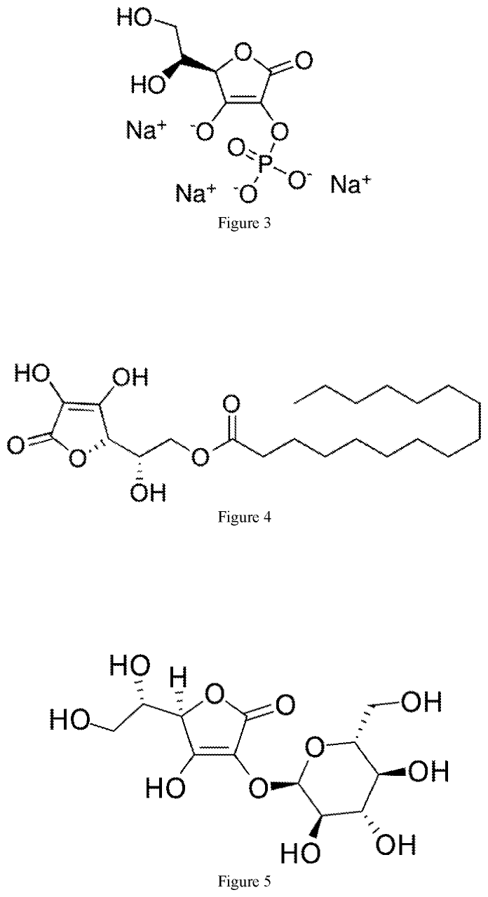

### DETAILED DESCRIPTION OF THE INVENTION

The Applicant describes the invention and its benefits in more detail in the following.

### Topical Detergent Composition

A first object of the invention is the topical detergent composition comprising
(i) ascorbyl glucoside and ascorbyl palmitate,
(ii) a washing and transcutaneous absorption-promoting base comprising
   at least one non-ionic surfactant,
   at least one anionic surfactant,
(iii) glycerol,
in combination with suitable excipients and/or diluents.

It should be noted that the composition of the invention is not in encapsulated form; preferably, the topical detergent composition is not in emulsion form.

It should further be noted that it is not necessary to treat the skin area onto which the topical detergent composition is applied, before or after application, with techniques such as: techniques using lasers, microdermabrasion, sonophoresis.

### Ascorbyl Glucoside and Ascorbyl Palmitate

Ascorbyl glucoside and ascorbyl palmitate are the two active agents, in precursor form of ascorbic acid, comprised in the topical detergent composition.

Preferably, the amount of ascorbyl glucoside is comprised between about 0.01% and 12%, preferably between about 0.05% and 12%, preferably between about 0.1% and 12%, preferably between about 0.5% and 12%, preferably between about 0.5% and 10%, preferably between about 0.5% and 8%, preferably between about 0.5% and 6%, preferably between about 0.5% and 5%, preferably between about 0.5% and 4%, preferably between about 1% and 4%, preferably about 1%, 2%, 3%, 4% by weight on the total weight of the composition.

Preferably, the amount of ascorbyl palmitate is comprised between about 0.01% and 12%, preferably between about 0.01% and 10%, preferably between about 0.01% and 5%, preferably between about 0.01% and 3%, preferably between about 0.05% and 3%, preferably between about 0.1% and 3%, preferably between about 0.2% and 3%, preferably between about 0.2% and 2%, preferably between about 0.2% and 1.5%, preferably between about 0.2% and 1%, preferably between about 0.2% and 0.7%, preferably between about 0.2% and 0.6%, preferably between about 0.4% and 0.6% by weight on the total weight of the composition.

It should be noted that ascorbyl palmitate not only constitutes a source of ascorbic acid/ascorbate, but also reinforces the detergent action of the washing base.

Preferably, the ascorbyl glucoside and/or ascorbyl palmitate are not in encapsulated or vesicular form.

Preferably, the topical detergent composition does not contain ascorbic acid as such or phosphate derivatives of ascorbic acid, such as sodium or magnesium ascorbyl phosphate.

### Washing and Transcutaneous Absorption-Promoting Base

Washing and transcutaneous absorption-promoting base (or simply washing base) is intended as a mixture of at least two surfactants of different chemical nature having a washing or detergent action on the skin surface, but also a transcutaneous absorption-promoting action, preferably of the ascorbyl glucoside and/or palmitate.

The washing and transcutaneous absorption-promoting base comprises
at least one non-ionic surfactant,
at least one anionic surfactant.

Preferably, the washing base comprises
at least two, preferably three, preferably four, non-ionic surfactants,
at least one anionic surfactant, preferably between 1 and 3 anionic surfactants.

According to a further embodiment, preferred when the topical detergent composition is in liquid form, the washing base comprises
at least one non-ionic surfactant, preferably between 1 and 4,
at least one anionic surfactant, preferably between 1 and 3,
at least one amphoteric surfactant, preferably one.

Preferably, the washing base does not contain any cationic surfactant, regardless of whether the topical detergent composition is made in liquid or solid form.

The amount of the washing base is preferably comprised between about 10% and 60%, preferably between about 20% and 60%, preferably between about 30% and 60%, preferably between about 35% and 60% by weight on the total weight of the composition.

When the detergent composition is in solid form, the amount of washing base is preferably comprised between about 35% and 60%, preferably between about 40% and 60%, preferably between about 50% and 60%, preferably between about 55% and 60%, preferably between about 57% and 60%, preferably about 58%, 59% or 59.5% by weight on the total weight of the composition.

When the detergent composition is in liquid form, the amount of washing base is preferably comprised between about 35% and 50%, preferably between about 35% and 45%, preferably about 36%, 37%, 38%, 39%, 40%, 41%, 42% or 43% by weight on the total weight of the composition.

Preferably, the non-ionic surfactant is selected from the group consisting of (indicated by the corresponding INCI names): decyl polyglucose, lauryl polyglucose, decyl glucoside, coco glucoside, lauryl glucoside, caprylyl/capryl glucoside, polyglyceryl-3 cocoate, polyglyceryl-6 caprylate, polyglyceryl-4 caprate, polyglyceryl-6 ricinoleate, polysorbate-20, polysorbate-80, peg-7 glyceryl cocoate, and mixtures of the foregoing.

Preferably, the ionic surfactant is selected from the group consisting of (indicated by the corresponding INCI names): disodium laureth sulfosuccinate, disodium ricinoleamide mea sulfosuccinate, sodium cocoyl glutamate, disodium cocoyl glutamate, sodium lauroyl glutamate, sodium methylcocoyl taurate, sodium cocoyl isethionate, sodium lauroyl sarcosinate, potassium cocoyl hydrolyzed collagen, sodium cocoyl hydrolyzed wheat protein, sodium lauryl sulfoacetate, sodium cocopolyglucose citrate, sodium coco-glucoside tartrate, olivoyl fructoside, olivoyl glutamate, and mixtures of the foregoing.

Preferably, the amphoteric surfactant is selected from the group consisting of (indicated by the corresponding INCI names): cocamido propyl betaine, lauramido propyl betaine, disodium cocoamphodiacetate, sodium cocoamphoacetate, sodium lauroamphoacetate, sodium olivamphoacetate, and mixtures of the foregoing.

Preferably, the weight ratio between the ascorbyl glucoside and the washing base is comprised between 1:10 and 1:70, preferably between 1:10 and 1:65, preferably between 1:10 and 1:60, preferably between 1:12 and 1:60, preferably between 1:12 and about 1:59.5, preferably between 1:12 and 1:50, preferably between 1:12 and 1:40, preferably between 1:12 and 1:30, preferably between 1:12 and 1:25, preferably between 1:12 and 1:22, preferably between 1:12 and 1:21.5.

Preferably, the Applicant considers such a weight ratio functional for obtaining ascorbic acid permeation, and at the same time minimising the lipid-protein alteration of the stratum corneum.

### Glycerol

Preferably, the glycerol is in an amount comprised between about 1% and 10%, preferably between about 1% and 8%, preferably between about 1% and 7%, preferably between about 2% and 7%, preferably between about 3% and 6%, preferably about 3%, 4%, 6% by weight on the total weight of the composition.

It should be noted that glycerol acts as a humectant and counteracts skin dehydration.

Suitable Excipients and/or Diluents

The topical detergent composition contains suitable excipients and/or diluents; such excipients and/or diluents are those known to the person skilled in the art for the preparation of a topical detergent composition in liquid or solid form.

By way of example, the excipients and/or diluents can be selected from the group consisting of: gelling agents, pH modifiers, anti-foaming agents, conditioning agents, preservatives, perfumers, colourants.

An example of a gelling agent is preferably that selected from the group consisting of (indicated by corresponding INCI names): carbomer, hydroxyethylcellulose, xanthan gum, PEG-120 Methyl Glucose Dioleate, and mixtures of the foregoing.

An example of a pH modifier is preferably that selected from the group consisting of (indicated by corresponding INCI names): citric acid, ethanolamine, triethanolamine, L-arginine, sodium hydroxide, and mixtures of the foregoing.

An example of an anti-foaming agent is preferably selected from the group consisting of (indicated by corresponding INCI names): dimethicone, trimethicone, polydimethylsiloxane, and mixtures of the foregoing.

An example of a conditioning agent is preferably selected from the group consisting of (indicated by corresponding INCI names): polyquaternium-10, polyquaternium-4, hydrolized collagen, hydrolized keratin, and mixtures of the foregoing.

An example of a preservative is preferably selected from the group consisting of (indicated by corresponding INCI names): phenoxyethanol, benzoic acid, dehydroacetic acid, capryloyl glycine, undecylenic acid, and mixtures of the foregoing.

### Lipid-Ceramidic Complex

The composition can also comprise a lipid-ceramidic complex comprising:
at least one ceramide,
at least cholesterol or a derivative thereof,
at least one saturated or unsaturated amino alcohol precursor of cell membrane sphingolipids.

The at least one ceramide is selected from the group consisting of: Ceramide NP, Ceramide AP, Ceramide EOP, and mixtures of the foregoing.

At least cholesterol or a derivative thereof is selected from the group consisting of: cholesterol, cholesterol sulphate, and mixtures of the foregoing.

The at least one saturated or unsaturated aminoalcohol precursor of cell membrane sphingolipids has a number of carbon atoms comprised between C15 and C20, preferably comprised between C17 and C19, preferably it is C18. Preferably, it is selected from the group consisting of: sphingosine, phytosphingosine, sphinganine, and mixtures of the foregoing, preferably it is phytosphingosine.

Preferably, the amount of the lipid-ceramidic complex is comprised between about 0.01% and 5%, preferably between about 0.05% and 5%, preferably between about 0.1% and 5%, preferably between about 0.1% and 3%, preferably between about 0.1% and 1.5%, preferably between about 0.1% and 1%, preferably between about 0.1% and 0.8%, preferably between about 0.2% and 0.8% by weight on the total weight of the composition.

Preferably, the lipid-ceramidic complex consists of
at least one ceramide,
at least cholesterol or a derivative thereof,
at least one saturated or unsaturated amino alcohol precursor of cell membrane sphingolipids.
an anionic surfactant
a thickening and/or viscosifying agent, and/or
a stabiliser.

An example of a surfactant, preferably an anionic surfactant, is Sodium Lauroyl Lactylate.

An example of a thickening or viscosifying agent or stabiliser is Carbomer and/or Xanthan Gum.

The Applicant considers that the lipid-ceramidic complex possesses an emollient function, capable of counteracting alteration phenomena of the skin barrier, caused by external agents and/or by the washing base comprised in the composition of the invention. The lipid-ceramidic complex also has a stratum corneum biomimicry effect, given the structural similarity with the latter, i.e., it can mimic the composition of the human skin barrier, preferably stratum corneum lipids; in this sense, it further promotes the transcutaneous permeation of ascorbyl, in the form of glycoside and/or palmitate.

### Possible Active Agents

Preferably, the topical detergent composition can comprise active agents selected the group consisting of: wetting, soothing, re-epithelialising, keratolytic, depigmenting, dermo-trophic agents and mixtures of the foregoing.

Preferably, the topical detergent composition can comprise active agents selected from the group consisting of (corresponding INCI names): panthenol, tocopheryl acetate, urea, sodium PCA, hyaluronic acid, allantoin, glycolic acid, nicotinamide, acetylglucosamine, oligopeptide-68, diacetyl boldine, retinyl palmitate, bakuchiol, and mixtures of the foregoing.

Preferably, the topical detergent composition can comprise at least one active agent in an amount comprised between about 0.1% and 7%, preferably between about 0.3% and 7%, preferably between about 0.3% and 5%, preferably about 0.3%, 1.3%, 3%, 5% by weight on the total weight of the composition.

### Further Features and Form of the Topical Detergent Composition

The topical detergent composition is characterised by a pH comprised between 5 and 6.

The Applicant considers that the pH of the composition is obtained also considering the surfactants in the washing base.

It is known that compositions or solutions with a high pH (pH close to 10), even in the absence of surfactants, can increase swelling of the stratum corneum (SC) and alter lipid stiffness, inducing skin damage.

The topical detergent composition is in liquid or solid form.

According to a preferred form, when the topical detergent composition is in liquid form, the latter can comprise water. Although not intentionally added to the formula of the solid version of the topical detergent composition, water is also present in the latter embodiment of the invention (albeit in smaller or at least negligible amounts), as it is inherently present in some of the raw materials used.

In accordance with the liquid embodiment, the amount of water contained in the topical detergent composition is preferably comprised between about 20% and 60%, preferably between about 25% and 50%, preferably between about 25% and 47%, preferably between about 25% and 45%, preferably between about 30% and 45%, preferably between about 35% and 45%, preferably between about 40% and 45%, preferably between about 42% and 45%, preferably about 42%, 43%, 44%, 44.6%, or 45% by weight on the total weight of the composition.

It should be noted that the liquid topical detergent composition is effective despite the relatively high water content.

According to a preferred form, when the topical detergent composition is in solid form, the latter comprises at least one hydrogenated vegetable oil, preferably of a commercially available type.

At least one hydrogenated vegetable oil is defined as a vegetable oil, extracted from vegetable sources and known to a person skilled in the art, which has undergone a hydrogenation process, i.e., a process of partial or total saturation of the unsaturations which characterise the polyunsaturated fatty acids comprised in vegetable oils, preferably fatty acids with a number of carbon atoms comprised between C12 and C24. The hydrogenation process normally occurs using catalysts and at temperatures comprised between 120° C. and 210° C.

Preferably, the at least one hydrogenated vegetable oil is in solid or semi-solid form at room temperature, i.e., a temperature comprised between 25° C. and 27° C.

Preferably, the at least one hydrogenated vegetable oil is selected from the group consisting of (INCI names): Hydrogenated Vegetable Oil, Hydrogenated Rice Bran Oil, and mixtures of the foregoing.

The topical detergent composition can be made in the form of a cosmetic product or medical device.

A cosmetic product is defined as any substance or mixture intended to be applied to the external surfaces of the human body (epidermis, hair system and hair, nails, lips, external genital organs) or to the teeth and mucous membranes of the mouth for the purpose of essentially modifying and/or maintaining the aesthetic appearance, cleansing, perfuming. A cosmetic product has no medical benefit. A substance or mixture intended to be ingested, inhaled, injected or implanted into the human body is not considered a cosmetic product (Reference legislation: Regulation (EC) No. 1223/2009).

A ‘medical device’ is any instrument, apparatus, implant, substance or other product, whether used alone or in combination and intended by the manufacturer to be used in human beings for the diagnosis, prevention, control, therapy or alleviation of a disease; for the diagnosis, control, therapy, alleviation or compensation of an injury or handicap of study, replacement or modification of the anatomy or a physiological process; of intervention in conception, the product of which does not exert its principal action, in or on the human body, for which it is intended, by pharmacological or immunological means or by metabolic process but whose function may be assisted by such means (EU Regulation 745/2017).

### Use of the Topical Detergent Composition

The composition of the invention is for use in the treatment of a disorder or altered state of the skin.

Preferably, the composition can be used to treat skin ageing, or melanic pigmentations, or alterations of dermo-epidermal trophism, or conditions in which skin ageing, melanic pigmentations and alterations in dermo-epidermal trophism are present simultaneously.

Alterations in dermo-epidermal trophism are understood as phenomena such as
reduced biosynthesis of connective and extracellular matrix linked to mitochondrial impairment;
senescence of fibroblasts with increased gene expression of matrix-degrading enzymes.

### Preparation Process of the Topical Detergent Composition

The preparation process of the topical detergent composition in liquid form comprises the following steps:
a) combining the washing base, ascorbyl palmitate, suitable excipients and/or diluents, possibly adding at least one active agent, to obtain a first mixture;
b) separately, combining the glycerol, water, possibly also at least one active agent, then heating to a temperature comprised between 40° C. and 60° C., preferably 50° C., preferably leaving to stir for a time comprised between 10 and 20 minutes, preferably for 15 minutes, to obtain a gelled mixture, then allowing to cool;
c) combining the cooled gelled mixture with the first mixture under stirring to obtain a second mixture;
d) separately, arranging the ascorbyl glucoside, possibly with the addition of the lipid-ceramidic complex, and adding it to the second mixture, preferably together with water and other suitable excipients and/or diluents.

The preparation process of the topical detergent composition in solid form comprises the following steps:
a′) combining the washing base, ascorbyl palmitate and at least one hydrogenated vegetable oil, and melting, preferably at a temperature comprised between 70° C. and 90° C., preferably 80° C., to obtain a first mixture, possibly adding at least one active agent once a temperature comprised between 30° C. and 50° C., preferably 40° C., has been reached;
b′) separately, arranging the glycerol and ascorbyl glucoside to obtain a second mixture;
c′) combining the second mixture with the first mixture and any excipients and/or diluents, preferably at a temperature comprised between 30° C. and 50° C., preferably 40° C., and possibly adding the lipid-ceramidic complex.

### EXAMPLES

The Applicant provides the examples for merely illustrative and non-limiting purposes.

### Example 1—Topical Detergent Composition in Liquid Form

| | | | Dose × | Dose × |
|---|---|---|---|---|
| Phase | Raw material | INCI | 100 g | 300 g |
| A | PLANTACRAE 2000 | Decyl Glucoside | 12.00 | 36.00 |
| A | PLANTAPON LCG | Sodium Lauryl Glucose | 12.00 | 3600 |
| | | Carboxylate (and) Lauryl | | |
| | | Glucoside | | |
| A | PLANTAPON SF | Sodium Cocoamphoacetate (and) | 12.00 | 36.00 |
| | | Glycerin (and) Lauryl Glucoside | | |
| | | (and) Sodium Cocoyl Glutamate | | |
| | | (and) Sodium Lauryl Glucose | | |
| | | Carboxylate | | |
| A | Glucamate ™ DOE-120 | PEG-120 Methyl Glucose | 3.000 | 9.000 |
| | | Dioleate | | |
| B | TWEEN 80 | Polysorbate 80 | 4.000 | 12.00 |
| B | ASCORBYL | Ascorbyl palmitate | 0.400 | 1.200 |
| | PALMITATE | | | |
| B | TOCOPHERYL | Tocopheryl acetate | 1.300 | 3.900 |
| | ACETATE oil 67% | | | |
| B | EUXYL K 701 | Phenoxyethanol (and) Benzoic | 0.600 | 1.800 |
| | | Acid (and) Dehydroacetic Acid | | |
| C | GLYCEROL | Glycerol | 4.000 | 12.00 |
| C | PANTHENOL 70% | Panthenol | 3.000 | 9.000 |
| E | CITRIC ACID | Citric acid | 0.570 | 1.710 |
| E | SODIUM CITRATE | Sodium citrate | 2.070 | 6.210 |
| | DIHYDRATE | | | |
| E | DEMI WATER | Aqua | 10.00 | 30.00 |
| D | ASCORBYL | Ascorbyl glucoside | 2.000 | 6.000 |
| | GLUCOSIDE | | | |
| D | SKIN INFLUX V | Ceramide NP; Ceramide AP; | 0.200 | 0.600 |
| | | Ceramide EOP; | | |
| | | Phytosphingosine; Cholesterol; | | |
| | | Sodium Lauroyl Lactylate; | | |
| | | Carbomer; Xanthan Gum | | |
| F | PERFUME IPA FREE | Parfume | 0.300 | 0.900 |
| C | DEMI WATER | Aqua | q.s. 100 | q.s. 300 |

### Preparation Process of the Composition of Example 1:

Separately set up Phases A, B and C. Set up Phase A by mixing the three surfactants and the gelling agent PEG-120 Methyl Glucose Dioleate in glass beakers with very moderate stirring (avoid the development of foam). Set up Phase B by dispersing Vitamin E acetate and Euxyl 701 in Tween 80, moderately stirring until a clear, homogeneous, amber-coloured phase is obtained. Set up Phase C by mixing the Glycerol and Panthenol with water under stirring until a clear, colourless phase is obtained and then heating the phase to 50° C. on a hot plate. Let stir for at least 15 minutes and once a gelled, translucent, lump-free phase is obtained, proceed to cool to 25° C. Combine Phase B and Phase A under moderate stirring (avoid the formation of foam) and then combine Phase C, again under moderate stirring. Disperse the components of Phase D one at a time under moderate stirring and finally combine Phase E, adjusting the pH between 5.3 and 5.5 with the buffer solution of Citric Acid and Sodium Citrate (citrate buffer) and lastly add the perfume (Phase F).

### Example 2—Topical Detergent Composition in Liquid Form

| | | | Dose × | Dose × |
|---|---|---|---|---|
| Phase | Raw material | INCI | 100 g | 300 g |
| A | PLANTACRAE 2000 | Decyl Glucoside | 8.00 | 24.0 |
| A | PLANTAPON LCG | Sodium Lauryl Glucose Carboxylate | 16.0 | 48.0 |
| | | (and) Lauryl Glucoside | | |
| A | PLANTAPON SF | Sodium Cocoamphoacetate (and) | 6.00 | 18.0 |
| | | Glycerin (and) Lauryl Glucoside (and) | | |
| | | Sodium Cocoyl Glutamate (and) | | |
| | | Sodium Lauryl Glucose Carboxylate | | |
| A | Glucamate ™ DOE- | PEG-120 Methyl Glucose Dioleate | 3.00 | 9.00 |
| | 120 | | | |
| B | TWEEN 80 | Polysorbate 80 | 3.00 | 9.00 |
| B | Coenzyme Q10 | Ubiquinone | 0.30 | 0.90 |
| B | ASCORBYL | Ascorbyl palmitate | 0.600 | 1.800 |
| | PALMITATE | | | |
| B | EUXYL K 701 | Phenoxyethanol (and) Benzoic Acid | 0.60 | 1.80 |
| | | (and) Dehydroacetic Acid | | |
| C | GLYCEROL | Glycerol | 6.00 | 18.0 |
| C | UREA | Urea | 5.00 | 15.0 |
| E | CITRIC ACID | Citric acid | 0.62 | 1.86 |
| E | SODIUM CITRATE | Sodium citrate | 2.20 | 6.60 |
| | DIHYDRATE | | | |
| E | DEMI WATER | Aqua | 15.0 | 45.0 |
| D | ASCORBYL | Ascorbyl glucoside | 3.00 | 9.00 |
| | GLUCOSIDE | | | |
| D | SKIN INFLUX V | Ceramide NP; Ceramide AP; Ceramide | 0.80 | 2.40 |
| | | EOP; Phytosphingosine; Cholesterol; | | |
| | | Sodium Lauroyl Lactylate; Carbomer; | | |
| | | Xanthan Gum | | |
| F | PERFUME IPA | Parfume | 0.30 | 0.90 |
| | FREE | | | |
| C | DEMI WATER | Aqua | q.s. | q.s. |
| | | | at 100 | at 300 |

### Preparation Process of the Composition of Example 2:

Separately set up Phases A, B and C. Set up Phase A by mixing the three surfactants and the gelling agent PEG-120 Methyl Glucose Dioleate in glass beakers with very moderate stirring (avoid the development of foam). Set up Phase B by dispersing ubiquinone and Euxyl 701 in Tween 80, moderately stirring until a clear, homogeneous, amber-coloured phase is obtained. Set up Phase C by mixing the Glycerol and Urea with water under stirring until a clear, colourless phase is obtained and then heating the phase to 50° C. on a hot plate. Let stir for at least 15 minutes and once a gelled, translucent, lump-free phase is obtained, proceed to cool to 25° C. Combine Phase B and Phase A under moderate stirring (avoid the formation of foam) and then combine Phase C, again under moderate stirring. Disperse the components of Phase D one at a time under moderate stirring and finally combine Phase E, adjusting the pH between 5.3 and 5.5 with the buffer solution of Citric Acid and Sodium Citrate (citrate buffer) and lastly add the perfume (Phase F).

### Example 3—Topical Detergent Composition in Solid Form

| | | | Dose × | Dose × |
|---|---|---|---|---|
| Phase | Raw material | INCI | 100 g | 300 g |
| A | Hydrogenated | Hydrogenated Rice Bran Oil | 20.00 | 60.00 |
| | rice oil | | | |
| A | Hydrogenated | Hydrogenated Vegetable Oil | 15.00 | 45.00 |
| | vegetable oil | | | |
| A | Non-ionic | Polyglyceryl-4 Laurate | 7.500 | 22.50 |
| | surfactant | | | |
| A | Anionic | Sodium Cocoyl Isethionate | 52.00 | 156.0 |
| | surfactant | | | |
| A | Non-ionic | Ascorbyl palmitate | 1.000 | 3.000 |
| | surfactant | | | |
| B | Coenzyme Q10 | Ubiquinone | 0.300 | 0.900 |
| A | Preservative | Phenoxyethanol (and) Benzoic Acid (and) | 0.200 | 0.600 |
| | | Dehydroacetic Acid | | |
| C | GLYCEROL | Glycerol | 3.000 | 9.000 |
| C | ASCORBYL | Ascorbyl glucoside | 1.000 | 3.000 |
| | GLUCOSIDE | | | |
| D | SKIN INFLUX | Ceramide NP; Ceramide AP; Ceramide | 0.800 | 2.400 |
| | V | EOP; Phytosphingosine; Cholesterol; | | |
| | | Sodium Lauroyl Lactylate; Carbomer; | | |
| | | Xanthan Gum | | |
| E | PERFUME IPA | Parfume | 0.200 | 0.600 |
| | FREE | | | |

### Preparation Process of the Composition of Example 3:

Melt Phase A at 80° C. until a clear liquid phase is obtained. Once 40° C. is reached, disperse Phase B under stirring, followed by Phase C. Finally, add Phase D and Phase E and allow the mixture to congeal to room temperature.

### BIBLIOGRAPHY

1. Krutmann J, Schroeder P. Role of mitochondria in photoaging of human skin: the defective powerhouse model. J Investig Dermatol Symp Proc. 2009 August; 14(1):44-9. doi: 10.1038/jidsymp.2009.1
2. Quan T, Qin Z, Xia W, Shao Y, Voorhees J J, Fisher G J. Matrix-degrading metalloproteinases in photoaging. J Investig Dermatol Symp Proc. 2009 August; 14(1):20-4. doi: 10.1038/jidsymp.2009.8.
3. Gilchrest B A, Eller M S, Yaar M. Telomere-mediated effects on melanogenesis and skin aging. J Investig Dermatol Symp Proc. 2009 August; 14(1):25-31. doi: 10.1038/jidsymp.2009.9.
4. Krutmann J. Ultraviolet A radiation-induced biological effects in human skin: relevance for photoaging and photodermatosis. J Dermatol Sci. 2000 March; 23 Suppl 1:S22-6. doi: 10.1016/s0923-1811(99)00077-8.
5. Bernerd F, Passeron T, Castiel I, Marionnet C. The Damaging Effects of Long UVA (UVA1) Rays: A Major Challenge to Preserve Skin Health and Integrity. Int J Mol Sci. 2022 Jul. 26; 23(15):8243. doi: 10.3390/ijms23158243.
6. Kishimoto Y, Saito N, Kurita K, Shimokado K, Maruyama N, Ishigami A. Ascorbic acid enhances the expression of type 1 and type 4 collagen and SVCT2 in cultured human skin fibroblasts. Biochem Biophys Res Commun. 2013 Jan. 11; 430(2):579-84. doi: 10.1016/j.bbrc.2012.11.110.
7. Pinnell S R, Yang H, Omar M, Monteiro-Riviere N, DeBuys H V, Walker L C, Wang Y, Levine M. Topical L-ascorbic acid: percutaneous absorption studies. Dermatol Surg. 2001 February; 27(2):137-42. doi: 10.1046/j.1524-4725.2001.00264.x.
8. Stamford N P. Stability, transdermal penetration, and cutaneous effects of ascorbic acid and its derivatives. J Cosmet Dermatol. 2012 December; 11(4):310-7. doi: 10.1111/jocd.12006.
9. Abdulmajed K, Heard C M. Topical delivery of retinyl ascorbate co-drug. 1. Synthesis, penetration into and permeation across human skin. Int J Pharm. 2004 Aug. 6; 280(1-2):113-24. doi: 10.1016/j.ijpharm.2004.05.008.
10. Yamamoto I, Muto N, Murakami K, Akiyama J. Collagen synthesis in human skin fibroblasts is stimulated by a stable form of ascorbate, 2-O-alpha-D-glucopyranosyl-L-ascorbic acid. J Nutr. 1992 April; 122(4):871-7. doi: 10.1093/jn/122.4.871.
11. Melo-Guímaro S, Cardoso R, Paiva João C, Santos J, Melro E, Arnaut L G, Costa Pereira J, Serpa C. Efficient dermal delivery of ascorbic acid 2-glucoside with photoacoustic waves. Int J Cosmet Sci. 2022 August; 44(4):453-463. doi: 10.1111/ics.12793.
12. Jacques C, Genies C, Bacqueville D, Tourette A, Borotra N, Chaves F, Sanches F, Gaudry AL, Bessou-Touya S, Duplan H. Ascorbic acid 2-glucoside: An ascorbic acid pro-drug with longer-term antioxidant efficacy in skin. Int J Cosmet Sci. 2021 December; 43(6):691-702. doi: 10.1111/ics.12745.
13. Gopinath D, Ravi D, Rao B R, Apte S S, Renuka D, Rambhau D. Ascorbyl palmitate vesicles (Aspasomes): formation, characterization and applications. Int J Pharm. 2004 Mar. 1; 271(1-2):95-113. doi: 10.1016/j.ijpharm.2003.10.032.
14. Hornby S, Walters R, Tierney N, Appa Y, Dorfman G, Kamath Y. Effect of commercial cleansers on skin barrier permeability. Skin Res Technol. 2016 May; 22(2):196-202. doi: 10.1111/srt.12250. Epub 2015 Jun. 10.
15. Ananthapadmanabhan K P, Moore D J, Subramanyan K, Misra M, Meyer F. Cleansing without compromise: the impact of cleansers on the skin barrier and the technology of mild cleansing. Dermatol Ther. 2004; 17 Suppl 1:16-25. doi: 10.1111/j.1396-0296.2004.04s1002.x.
16. Schmid-Wendtner M H, Korting H C. The pH of the skin surface and its impact on the barrier function. Skin Pharmacol Physiol. 2006; 19(6):296-302. doi: 10.1159/000094670.

1. Topical detergent composition comprising
(i) ascorbyl glucoside and ascorbyl palmitate,
(ii) a washing and transcutaneous absorption-promoting base comprising
at least one non-ionic surfactant,
at least one anionic surfactant,
(iii) glycerol,
in combination with suitable excipients and/or diluents.

2. Composition according to claim 1, wherein the amount of ascorbyl glucoside is between 0.01% and 12% by weight on the total weight of the composition.

3. Composition according to claim 1, wherein the amount of ascorbyl palmitate is between 0.01% and 12% by weight on the total weight of the composition.

4. Composition according to claim 1, wherein the weight ratio between the ascorbyl glucoside and the washing base is between 1:10 and 1:70.

5. Composition according to claim 1, wherein the amount of glycerol is between 1% and 10% by weight on the total weight of the composition.

6. Composition according to claim 1, further comprising a lipid ceramidic complex comprising:
at least one ceramide,
at least cholesterol or a derivative thereof, and
at least one saturated or unsaturated amino alcohol precursor of cell membrane sphingolipids.

7. Composition according to claim 6, wherein the amount of the lipid-ceramidic complex is between 0.01% and 5% by weight on the total weight of the composition.

8. Composition according to claim 1, further comprising active agents selected from the group consisting of: wetting, soothing, re-epithelializing, keratolytic, depigmenting, dermo-trophic agents, and mixtures of the foregoing.

9. Composition according to claim 1, wherein the pH is between 5 and 6.

10. Composition according to claim 1, in the form of a cosmetic product or medical device.

11. Composition according to claim 1, in liquid or solid form.

12. Composition according to claim 11, comprising water, preferably in an amount between 20% and 60% by weight on the total weight of the composition.

13. Liquid composition according to claim 11, wherein the washing base also contains at least one amphoteric surfactant.

14. Solid composition according to claim 11, comprising at least one hydrogenated vegetable oil.

15. (canceled)

16. Method of treatment of a skin disorder or alternate state selected from skin ageing, or melanic pigmentation, or alteration of dermo-epidermal trophism or combinations of the foregoing using the composition according to claim 1.
